(19) United States
(12) Patent Application Publication
(10) Pub. No.: US 2022/0248939 A1
(43) Pub. Date: Aug. 11, 2022

(54) METHOD AND APPARATUS FOR ENDOSCOPIC DILATION

(71) Applicant: The University of Vermont and State Agricultural College, Burlington, VT (US)

(72) Inventors: Richard Zubarik, Williston, VT (US); Silas Hill, South Burlington, VT (US)

(73) Assignee: The University of Vermont and State Agricultural College, Burlington, VT (US)

(21) Appl. No.: 17/611,971

(22) PCT Filed: May 15, 2020
(86) PCT No.: PCT/US2020/033115

§ 371 Date: Nov. 17, 2021

Related U.S. Application Data
(60) Provisional application No. 62/849,519, filed on May 17, 2019.

(51) Int. Cl. A61B 1/00; A61M 25/10

(52) U.S. Cl. CPC A61B 1/00082; A61B 1/018; A61M 25/10188; A61M 25/10181

(57) ABSTRACT

Techniques for operating and monitoring an endoscopic balloon dilation system. The endoscopic balloon dilation system includes: an endoscope configured to be inserted into an esophagus of a patient; a balloon catheter comprising a balloon, the balloon catheter configured to pass through the endoscope into the esophagus of the patient; a balloon inflation device fluidly coupled to the balloon catheter, the balloon inflation device configured to dilate the balloon by flowing fluid into the balloon; a digital dilation monitor fluidly coupled to the balloon catheter and the balloon inflation device, the digital dilation monitor configured to measure at least one property of the balloon; a processor; and a non-transitory computer-readable storage medium storing processor executable instructions that, when executed by the processor, cause the processor to: receive, from the digital dilation monitor, information representing the at least one property of the balloon; and display, on a display device, a visual representation of the at least one property of the balloon.

### CROSS-REFERENCE TO RELATED APPLICATIONS

The present application claims the benefit of priority under 35 U.S.C. § 119(e) to U.S. Provisional Application Ser. No. 62/849,519, titled “METHOD AND APPARATUS FOR ENDOSCOPIC DILATION”, filed on May 17, 2019 (Attorney Docket No.: V0139.70128US00), which is incorporated by reference in its entirety.

### BACKGROUND

Balloon catheters may be used in a variety of systems in the body to dilate passageways such as vascular tissues, the sinus system, and the esophagus. For example, esophageal balloon dilation may be performed to stretch a narrowed portion of a patient's esophagus, to treat dysphagia or other conditions relating to strictures of the esophagus. Stretching a narrowed portion of the esophagus may improve passage of food, prevent impaction of food, improve nutrition, and improve ability to swallow.

In through-the-scope endoscopic balloon dilation, the balloon catheter may be passed through an endoscope into the passageway to be dilated. This may provide visual feedback during the balloon dilation procedure.

### SUMMARY

According to some embodiments, an endoscopic balloon dilation system is provided. The endoscopic balloon dilation may comprise: an endoscope configured to be inserted into an esophagus of a patient; a balloon catheter comprising a balloon, the balloon catheter configured to pass through the endoscope into the esophagus of the patient; a balloon inflation device fluidly coupled to the balloon catheter, the balloon inflation device configured to dilate the balloon by flowing fluid into the balloon; a digital dilation monitor fluidly coupled to the balloon catheter and the balloon inflation device, the digital dilation monitor configured to measure at least one property of the balloon; at least one processor; and at least one non-transitory computer-readable storage medium storing processor executable instructions. When executed by the at least one processor, the processor executable instructions may cause the at least one processor to perform a method comprising: receiving, from the digital dilation monitor, information representing the at least one property of the balloon; and displaying, on a display device, a visual representation of the at least one property of the balloon.

According to some embodiments, the at least one property of the balloon may comprise a pressure of the balloon.

According to some embodiments, the at least one property of the balloon may comprise a volume of the balloon.

According to some embodiments, the method may further comprise storing the information representing the at least one property of the balloon on the at least one non-transitory computer-readable storage medium.

According to some embodiments, the visual representation of the at least one property of the balloon may comprise a plot of the pressure of the balloon.

According to some embodiments, the visual representation of the at least one property of the balloon may comprise a plot of the volume of the balloon.

According to some embodiments, the visual representation of the at least one property of the balloon may be displayed to a user as part of a user interface.

According to some embodiments, the user interface may allow the user to select to store the information representing the at least one property of the balloon on the at least one non-transitory computer-readable storage medium

According to some embodiments, the method may further comprise calculating an energy of the system at a time based on the pressure and the volume of the balloon at the time.

According to some embodiments, the method may further comprise calculating a power input of the system at the time based on the energy and the time.

According to some embodiments, the method may further comprise receiving, from a user, a target pressure for the balloon.

According to some embodiments, the method may further comprise establishing, based on the target pressure, an upper control limit pressure for the balloon.

According to some embodiments, the method may further comprise establishing, based on the target pressure, a lower control limit pressure for the balloon.

According to some embodiments, the method may further comprise notifying the user if the pressure exceeds the upper control limit pressure for the balloon.

According to some embodiments, the method may further comprise notifying the user if the pressure is below the lower control limit pressure for the balloon.

According to some embodiments, notifying the user may comprise displaying a visual indicator to the user via the user interface.

According to some embodiments, the method may further comprise conditionally operating the balloon inflation device to dilate the balloon by flowing fluid into the balloon.

According to some embodiments, the balloon inflation device may be conditionally operated based on the information representing the at least one property of the balloon.

According to some embodiments, the balloon inflation device may be conditionally operated based on a target pressure.

According to some embodiments, the digital dilation monitor may be configured to measure the at least one property of the balloon in real time as the balloon is being dilated.

According to some embodiments, the visual representation of the at least one property the balloon may be displayed in real time as the balloon is being dilated.

According to some embodiments, a method of performing endoscopic balloon dilation using a digital dilation monitor is provided. The method may comprise: fluidly coupling the digital dilation monitor to a balloon catheter and a balloon inflation device, the balloon catheter configured to pass through an endoscope into an esophagus of a patient; operating the balloon inflation device to dilate the balloon of the balloon catheter to a first pressure by flowing fluid into the balloon; measuring, using the digital dilation monitor, a pressure and a volume of the balloon; and monitoring the pressure and the volume of the balloon based on a visual representation of the pressure and the volume, the visual representation displayed on a display device.

According to some embodiments, monitoring the pressure and the volume may occur in real time while dilating the balloon.

According to some embodiments, monitoring the pressure and the volume of the balloon may comprise monitoring a pressure-volume graph.

According to some embodiments, the method may further comprise conditionally dilating the balloon to a second pressure based at least in part on the monitored pressure and volume of the balloon.

According to some embodiments, the method may further comprise selecting, via a user interface displayed on the display device, to store information representing the pressure and the volume of the balloon on at least one non-transitory computer-readable storage medium.

According to some embodiments, the method may further comprise setting, via a user interface displayed on the display device, at least one control limit for the pressure or the volume of the balloon.

According to some embodiments, monitoring the pressure and the volume of the balloon may further comprise determining whether the balloon inflation device should be replaced.

According to some embodiments, at least one non-transitory computer-readable storage medium is provided, storing processor executable instructions that, when executed by at least one processor, may cause the processor to perform a method comprising: receiving, from a digital dilation monitor, information representing at least one property of a balloon of a balloon catheter, the digital dilation monitor fluidly coupled to the balloon catheter and a balloon inflation device, the balloon catheter configured to pass through an endoscope into an esophagus of a patient, the balloon inflation device configured to dilate the balloon by flowing fluid into the balloon; and displaying, on a display device, a visual representation of the at least one property of the balloon.

According to some embodiments, the at least one property of the balloon may comprise a pressure of the balloon.

According to some embodiments, the at least one property of the balloon may comprise a volume of the balloon.

According to some embodiments, the method may further comprise storing the information representing the at least one property of the balloon on the at least one non-transitory computer-readable storage medium.

According to some embodiments, the visual representation of the at least one property of the balloon may comprise a plot of the pressure of the balloon.

According to some embodiments, the visual representation of the at least one property of the balloon may comprise a plot of the volume of the balloon.

According to some embodiments, the visual representation of the at least one property of the balloon may be displayed to a user as part of a user interface.

According to some embodiments, the user interface may allow the user to select to store the information representing the at least one property of the balloon on the at least one non-transitory computer-readable storage medium.

According to some embodiments, the method may further comprise calculating an energy of the system at a time based on the pressure and the volume of the balloon at the time.

According to some embodiments, the method may further comprise calculating a power input of the system at the time based on the energy and the time.

According to some embodiments, the method may further comprise receiving, from a user, a target pressure for the balloon.

According to some embodiments, the method may further comprise establishing, based on the target pressure, an upper control limit pressure for the balloon.

According to some embodiments, the method may further comprise establishing, based on the target pressure, a lower control limit pressure for the balloon.

According to some embodiments, the method may further comprise notifying the user if the pressure exceeds the upper control limit pressure for the balloon.

According to some embodiments, the method may further comprise notifying the user if the pressure is below the lower control limit pressure for the balloon.

According to some embodiments, notifying the user may comprise displaying a visual indicator to the user via the user interface.

According to some embodiments, the method may further comprise conditionally operating the balloon inflation device to dilate the balloon by flowing fluid into the balloon.

According to some embodiments, the balloon inflation device may be conditionally operated based on the information representing the at least one property of the balloon.

According to some embodiments, the balloon inflation device may be conditionally operated based on a target pressure.

According to some embodiments, the digital dilation monitor may be configured to measure the at least one property of the balloon in real time as the balloon is being dilated.

According to some embodiments, the visual representation of the at least one property the balloon may be displayed in real time as the balloon is being dilated.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 depicts an illustrative endoscope, in accordance with some embodiments of the technology described herein;

FIG. 2 depicts an illustrative balloon catheter, in accordance with some embodiments of the technology described herein;

FIG. 3 depicts an illustrative pump for a balloon catheter, in accordance with some embodiments of the technology described herein;

FIG. 4 depicts an illustrative digital dilation monitor, in accordance with some embodiments of the technology described herein;

FIGS. 5A-5B depict illustrative images from endoscopic balloon dilation, in accordance with some embodiments of the technology described herein;

FIG. 6A-B depict, schematically, an illustrative endoscopic dilation system including a digital dilation monitor, in accordance with some embodiments of the technology described herein;

FIG. 7 is a flow diagram illustrating a method for operating an endoscopic dilation system including a digital dilation monitor, in accordance with some embodiments of the technology described herein;

FIG. 8 is a flow diagram illustrating a computer-implemented method for displaying information from an endoscopic dilation system, in accordance with some embodiments of the technology described herein;

FIG. 9 depicts an illustrative user interface for an endoscopic dilation system, in accordance with some embodiments of the technology described herein;

FIG. 10 is a flow diagram illustrating a computer-implemented method for operating an endoscopic dilation system, in accordance with some embodiments of the technology described herein; and

FIG. 11 is a block diagram of an illustrative computer system that may be used in implementing some embodiments of the technology described herein.

### DETAILED DESCRIPTION

Balloon catheter dilation procedures may be useful for treating conditions affecting a variety of systems in the body, including the vascular system, the sinus system, and/or the esophageal system. In particular, endoscopic balloon dilation procedures may be used to treat conditions such as dysphagia. By inserting a balloon catheter into the esophagus and inflating it, strictures affecting esophageal function may be dilated, relieving symptoms.

However, the inventors have recognized and appreciated that repeated esophageal dilation procedures may be required to fully dilate esophageal strictures, and serious complications such as esophageal punctures may occur. The inventors have further recognized and appreciated that conventional methods of balloon catheter dilation typically comprise inflating the balloon with little or no feedback about balloon diameter, volume, and/or pressure. In general, conventional techniques have not provided means to collect, monitor, and/or record accurate quantitative data (such as balloon diameter, volume, and pressure) during endoscopic balloon dilation procedures. This may lead to ineffective treatment methods (e.g., requiring multiple dilation procedures and/or failing to relieve the patient's symptoms) or cause active harm to the patient (e.g., mucosal damage).

The inventors have also recognized that, with conventional techniques, the specific dilating balloon chosen for dilation may be empiric. For example, after inserting an endoscope into the esophagus of the patient and passing a balloon catheter through the endoscope, a physician may select a size of dilating balloon empirically (e.g., based on a visual evaluation of a stricture of the patient's esophagus). The empiric nature of balloon selection for dilation may increase the likelihood of under dilation (e.g., failing to inflate the balloon to a desired diameter or volume) or over dilation (e.g., inflating the balloon past a desired diameter or volume) during a balloon dilation procedure. For example, with esophageal balloon dilation, if the narrowing of the esophagus is under dilated, then the patient undergoing the procedure may need to be subjected to multiple procedures to achieve the desired clinical outcome. If the narrowing of the esophagus is over dilated, then there is an increased risk of mucosal damage or esophageal perforation, which is a potentially life threatening condition.

The inventors have also recognized and appreciated that, with conventional techniques, the physical process of inflating the balloon (e.g., with a balloon inflation device, such as a hand-operated pump having a trigger or crank mechanism) is generally not performed by the physician supervising the procedure, but rather by an assistant (e.g., a nurse or technician). As recognized and appreciated by the inventors, conventional techniques for endoscopic balloon dilation do not provide any means for accurately determining whether such an assistant is executing inflation of the balloon to the desired parameters set by the physician (e.g., due a lack of quantitative feedback during the procedure). As a result, endoscopic balloon dilation procedures may be executed with a lower precision than desired, increasing the risk of ineffective procedures and/or harm to the patient.

Consequently, the inventors have developed and implemented techniques for monitoring balloon catheter inflation during through-the-scope endoscopic dilation procedures. The inventors have recognized and appreciated that receiving feedback (e.g., data regarding properties of the balloon, such as balloon diameter, volume, or pressure) during a dilation procedure may affect patient outcomes, including limiting or reducing mucosal damage, preventing life-threatening punctures, and/or reducing the number of procedures required to complete treatment. In contrast with conventional techniques (e.g., where the dilation may be performed by an assistant, with little or no feedback regarding the properties of the balloon), the techniques described herein provide the physician with accurate, real-time feedback on the precise quantitative properties of the balloon during the dilation process. As a result, the techniques described herein may provide the physician with substantially more oversight and control of the dilation process. This may provide a number of benefits, such as increasing the efficacy of dilation, reducing the need for repeated dilation procedures, and/or reducing the risk of physical harm to the patient during the dilation.

Moreover, the collection of precise quantitative data during endoscopic balloon dilation according to the techniques described herein may allow aspects of endoscopic balloon dilation to be performed automatically, with little or no human intervention, with a high degree of accuracy. For example, with the techniques described herein, a balloon inflation device may be automatically operated by a computer based on the real-time, quantitative data collected regarding the properties of the balloon. The techniques described herein may also allow patient-specific data collection to be leveraged in order to improve the efficacy of repeated dilation procedures. For example, if a particular patient's condition responds well to balloon catheter dilation with particular balloon properties (e.g., a particular pressure/volume, or particular pressure/volume curves during dilation), then those properties can be precisely replicated during future dilation procedures. If a particular patient's condition is not improved by balloon catheter dilation, then more aggressive balloon properties (e.g., increased pressure/volume) can be selected for future dilation procedures.

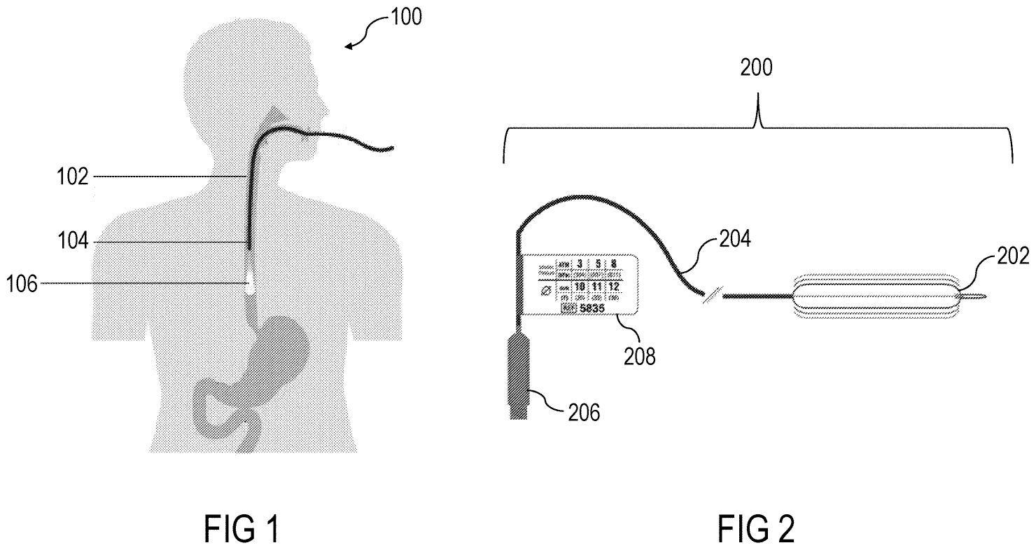

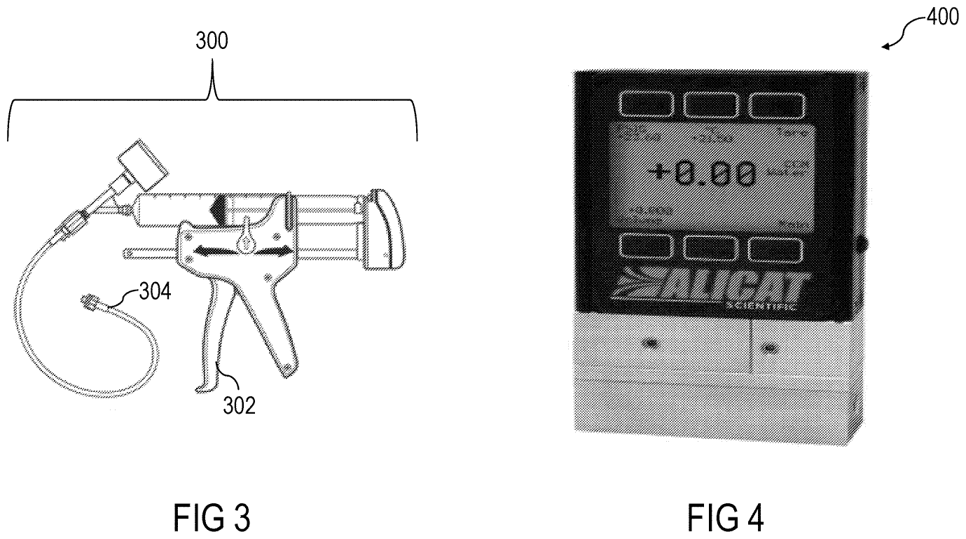

As described herein, the techniques discovered by the inventors provide substantial improvements to existing techniques for through-the-scope endoscopic balloon dilation. Accordingly, some embodiments of the technology described herein include an endoscopic balloon dilation system. The endoscopic balloon dilation system may include an endoscope (e.g., such as the one shown in FIG. 1) which can be inserted into an esophagus (or other bodily passageway) of a patient. The system may further include a balloon catheter (e.g., a wire-guided balloon catheter, such as the one shown in FIG. 2) with a balloon (e.g., a controlled radial expansion (CRE) balloon). The balloon catheter can pass through the endoscope into the esophagus (or other bodily passage) of the patient. The system may include a balloon inflation device (e.g., a pump, such as an automatic or manually powered pump, such as the one shown in FIG. 3), which can be fluidly coupled (e.g., with tubing of an appropriate diameter) to the balloon catheter. The balloon inflation device may be operable to dilate the balloon by flowing fluid (e.g., saline) into the balloon. According to some embodiments, the system may also include a digital dilation monitor (e.g., any suitable digital sensing device, including but not limited to a digital manometer, such as the one shown in FIG. 4) fluidly coupled (e.g., with tubing of an appropriate diameter) to the balloon catheter and the balloon inflation device. The digital dilation monitor may be configured to measure (e.g., in real time) at least one property of the balloon (e.g., a pressure, volume, diameter, and/or any other suitable property of the balloon)

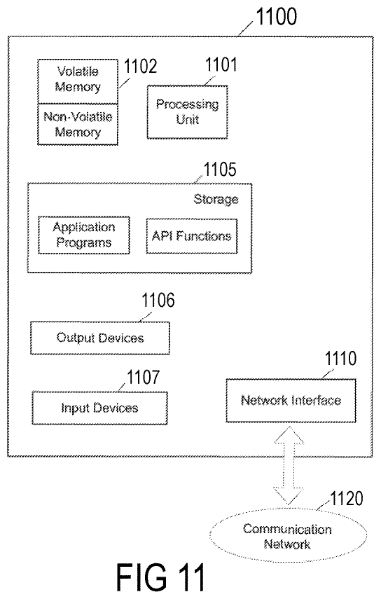

In some embodiments, the system may further include one or more computing devices, having at least one processor and at least one non-transitory computer-readable storage medium storing processor executable instructions (see, e.g., FIG. 11). When the processor executable instructions are executed by the at least one processor, they may cause the at least one processor to perform a method comprising: receiving (e.g., via a wired or wireless communication channel) information representing the property of the balloon (e.g., numeric values representing the pressure, volume, diameter, and/or any other suitable property of the balloon) from the digital dilation monitor; and displaying (e.g., in real time) a visual representation (e.g., a plot or other graphic, a counter with one or more numeric values, etc.) of the property of the balloon on a display device (e.g., a laptop or other device screen, a standalone monitor, or any other suitable display device). The method carried out by the processor may further comprise calculating an energy of the system at a particular time, based on the pressure and the volume of the balloon at that time. The method may also include calculating a power input of the system at that time based on the energy.

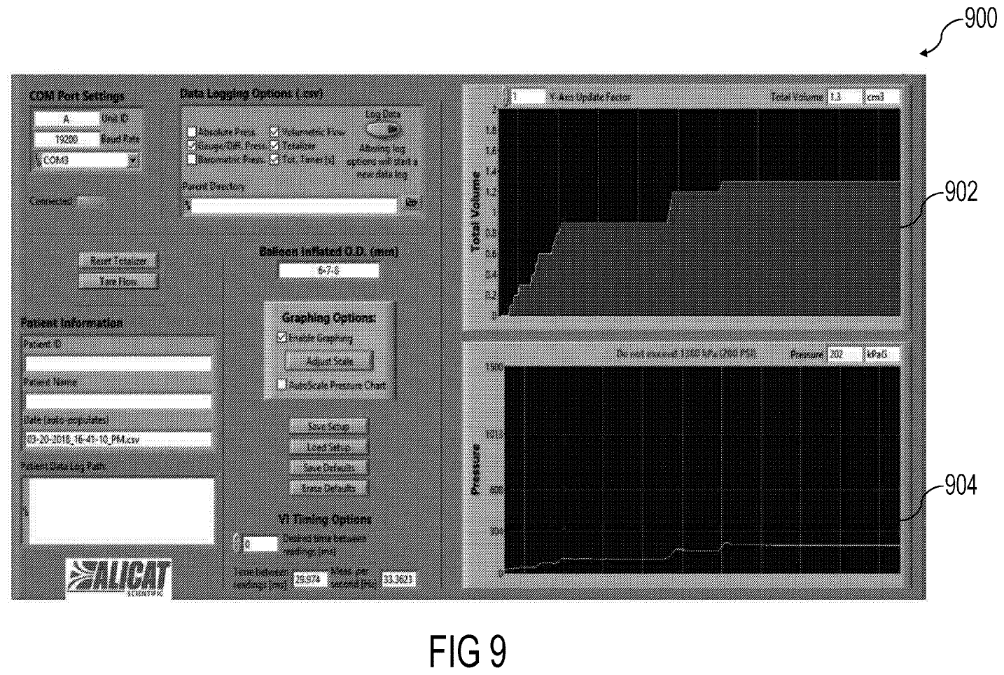

According to some embodiments, the information representing the property of the balloon may be stored on the at least one non-transitory computer-readable storage medium. In some embodiments, the property of the balloon may be displayed to a user as part of a user interface (e.g., a graphical user interface, such as the one shown in FIG. 9). The user interface may allow the user to select to store the information representing the property of the balloon (e.g., on the at least one non-transitory computer-readable storage medium). In some embodiments, the method carried out by the process may further include receiving from a user (e.g., by typing into the user interface, or supplying to the system in any other suitable manner) a target pressure (and/or target diameter or another target property) for the balloon. Based on the target pressure, an upper control limit pressure and/or lower control limit pressure (or corresponding thresholds for any suitable property) for the balloon may be established. In some embodiments, the method carried out by the processor may include notifying the user (e.g., with any suitable audio or visual indicator, such as a light or a pop-up on the display device, a warning tone through the speakers, etc) if the pressure exceeds the upper control limit pressure or falls below the lower control limit pressure.

In some embodiments, the method carried out by the processor may include conditionally operating the balloon inflation device (e.g., a computer-operable pump) to dilate the balloon by flowing fluid into the balloon. The balloon inflation device may be conditionally operated based on the information representing the property of the balloon (e.g., in response to real-time measurements from the digital dilation monitor) as well as a target pressure (or other target property) for the balloon.

According to some embodiments of the techniques described herein, a method of performing endoscopic balloon dilation using a digital dilation monitor is provided. The method may include fluidly coupling (e.g., with tubing and connectors to facilitate fluid flow) the digital dilation monitor to a balloon catheter and a balloon inflation device. As noted elsewhere herein, the balloon catheter may configured to pass through an endoscope into an esophagus of a patient. The method may further include operating the balloon inflation device (e.g., by operating a trigger or cranking mechanism) to dilate the balloon of the balloon catheter to a first pressure by flowing fluid into the balloon. The method may also include measuring, using the digital dilation monitor, a pressure and a volume of the balloon, and monitoring (e.g., in real time) the pressure and the volume of the balloon based on a visual representation of the pressure and the volume on a display device.

In some embodiments, monitoring the pressure and the volume of the balloon comprises monitoring a pressure-volume graph, or monitoring the pressure and the volume to determine whether the balloon inflation device or balloon should be replaced. In some embodiments, the method may further include conditionally dilating the balloon to a second pressure based at least in part on the monitored pressure and volume of the balloon. In some embodiments, the method may further include selecting, via a user interface displayed on the display device, to store information representing the pressure and the volume of the balloon on at least one non-transitory computer-readable storage medium. The method may also include setting, via a user interface displayed on the display device, at least one control limit for the pressure or the volume of the balloon.

Following below are more detailed descriptions of various concepts related to, and embodiments of, techniques for endoscopic dilation. It should be appreciated that various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

FIG. 1 depicts an endoscope 104, in accordance with some embodiments of the techniques described herein. As shown in the depicted example, the endoscope 104 may be inserted into the esophagus 102 of a patient 100. A light 106 at the end of the endoscope allows images of the internal anatomy of the patient 100 to be transmitted back to an observer. In the depicted example, the endoscope 104 passes through the mouth and throat of the patient, into esophagus 102. The endoscope 104 may provide images of the esophagus 102, as well as images of the stomach and upper small intestine. In some cases, other passageways within the body of patient 100 may be imaged using an endoscope 104 (e.g., the colon and large intestine, the sinus system, etc). In some embodiments, the endoscope 104 may comprise a tube having a hollow center (e.g., wide enough to allow passage of a balloon catheter).

FIG. 2 depicts a balloon catheter 200, in accordance with some embodiments of the techniques described herein. Balloon catheter 200 may be any suitable balloon catheter for performing endoscopic dilation, as described herein. As shown in the depicted example, the balloon catheter 200 may comprise a balloon 202. The balloon 202 may be a controlled radial expansion (CRE) balloon, or any other suitable type of balloon for dilating an esophagus of a patient. The balloon 202 may be a wire-guided balloon, having a fixed guide wire that may allow the balloon 202 to pass more easily through the working channel (e.g., an endoscope or narrowed portion of the esophagus through which the balloon 202 must pass). As shown in the depicted example, the balloon catheter 200 may include a catheter 204, which can be, for example, tubing of any suitable diameter. The catheter 204 may end with a connector 206, which may be configured to attach to a pumping mechanism, a digital dilation monitor, or additional tubing, as described herein. In some examples, the balloon catheter may be a Boston Scientific CRE Fixed Wire Balloon Catheter, as shown.

The balloon catheter 200 may also include a label 208, which may have information about the balloon catheter 200, such as a reference number. In some cases, the label 208 may include a chart indicating an inflation pressure (e.g., 3, 5, or 8 ATM) and corresponding diameter of the balloon 202 (e.g., 10, 11, or 12 mm). Note that in some cases, the information on label 208 may not be accurate. For example, with repeated use, the balloon 202 may experience wear, resulting in changes to the correspondence between pressure and diameter as shown on the label 208. Differences in manufacturing, the environment of use, and other factors may also cause the actual functionality of the balloon catheter 200 to differ from the information shown on label 208, in some cases. The techniques described herein may serve to provide a more accurate means for characterizing and monitoring the inflation characteristics (e.g., a correspondence between a pressure and diameter and/or volume) of the balloon 202. In some examples, the techniques described herein may be used to develop a model (e.g., a statistical model) representing the inflation characteristics of the balloon 202.

FIG. 3 depicts a balloon inflation device 300, in accordance with some embodiments of the techniques described herein. Balloon inflation device 300 may require manual user input to flow fluid and dilate the balloon (e.g., such as balloon 202). In the depicted example, the balloon inflation device includes a trigger 302, which may be depressed in order to initiate fluid flow (e.g., of a saline or other sterile solution) through a tubing connection 304 of the balloon inflation device. In some embodiments, other mechanisms for operating the balloon inflation device 300 may be provided, such as a cranking mechanism. In some embodiments, the balloon inflation device need not be operated manually. For example, a computer-operable mechanism may be provided to allow a computing device to operate the balloon inflation device.

The tubing connection 304 of the balloon inflation device 300 may be configured to connect to a balloon catheter (e.g., using connector 206), a digital dilation monitor, or additional tubing, as described herein. The balloon inflation device 300 may be a Boston Scientific dilation pump with a 10″ tubing connection, as shown

FIG. 4 depicts a digital dilation monitor 400, in accordance with some embodiments of the techniques described herein. The digital dilation monitor 400 may be configured to measure one or more properties of a balloon of a balloon catheter (e.g., balloon 202 of balloon catheter 200). For example, the digital dilation monitor 400 may measure a pressure within the balloon, a volume of the balloon, a diameter of the balloon, or any other properties relation to the balloon and the inflation thereof. For example, the digital dilation monitor 400 may include a digital manometer, or any other suitable sensors capable of measuring fluid flow. In some embodiments, the digital dilation monitor 400 may be an Alicat Scientific L Series Flow Meter, as shown.

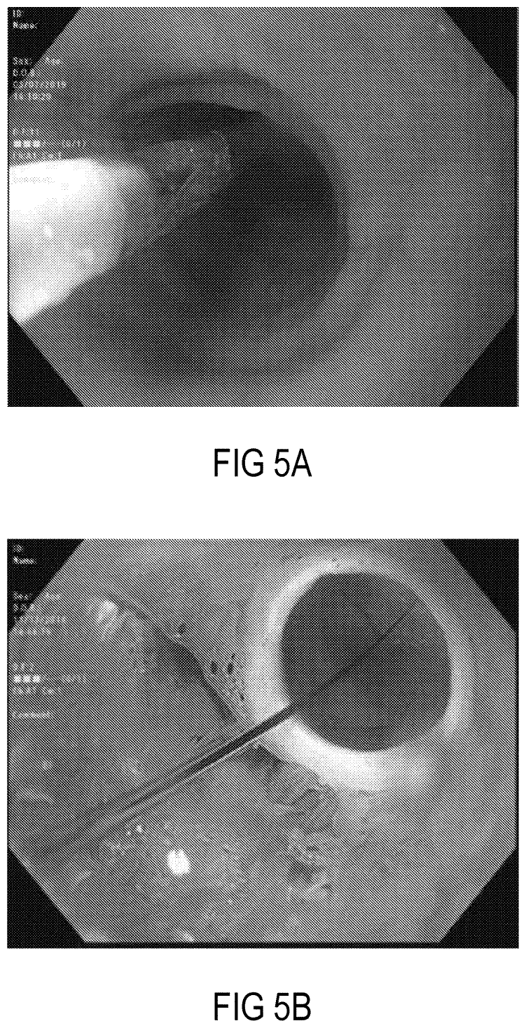

FIGS. 5A-5B depict illustrative images of an endoscopic balloon dilation procedure, in accordance with some embodiments of the technology described herein. In the depicted examples, the images are images of an esophagus of a patient, such as may be captured with an endoscope (e.g., as described herein at least with respect to FIG. 1) during an endoscopic dilation procedure. In the depicted examples, portions of a balloon catheter (e.g., as described herein at least with respect to FIG. 2) being used in the endoscopic dilation procedure are visible. As can be observed in the depicted example images, the inflation of the balloon of the balloon catheter results in the balloon expanding, dilating the esophagus of the patient.

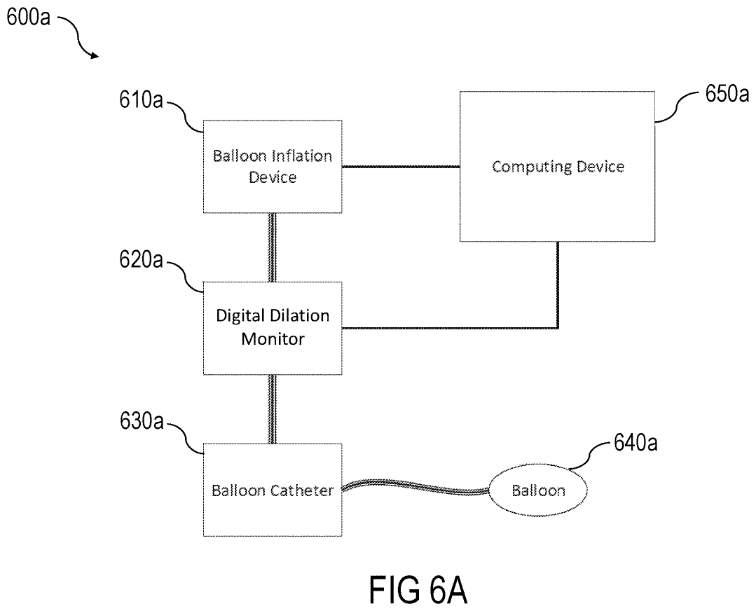

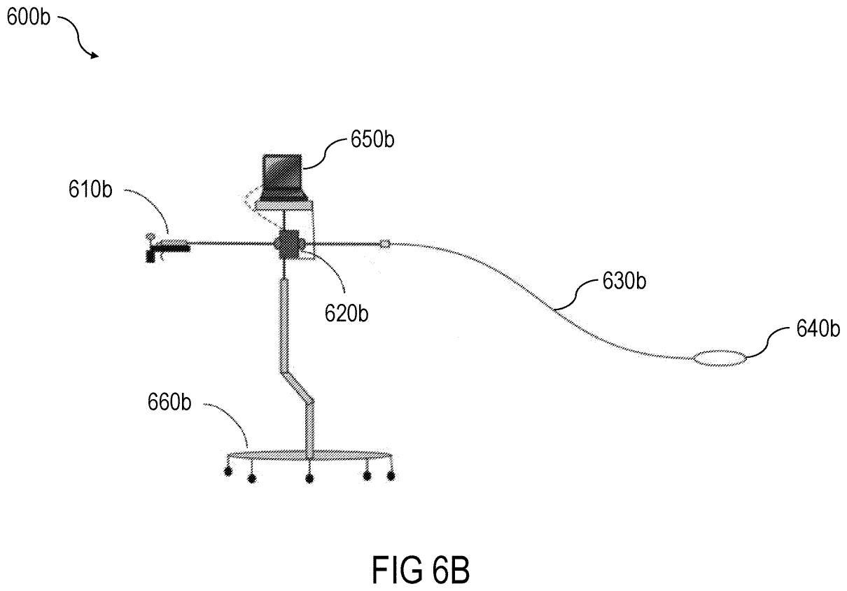

FIG. 6A depicts, schematically, an illustrative endoscopic dilation system 600a according to some embodiments. Endoscopic dilation system 600a may include a balloon inflation device 610a (e.g., as shown FIG. 3) fluidly coupled to a balloon catheter 630a. The balloon catheter 630a may include a balloon 640a fluidly coupled to balloon catheter 630a (e.g., as shown in FIG. 2). Digital monitor 620a may be fluidly coupled between the balloon inflation device 610a and balloon catheter 640a. Digital dilation monitor 620a may also be electronically coupled to computing device 650a (e.g., via a wired connection, a wireless connection, or any other suitable means). Computing device 650a may be any suitable kind of computing device, as discussed herein at least with respect to FIG. 11.

Balloon inflation device 610a may be any suitable device for endoscopic dilation, including but not limited to a balloon inflation device such balloon inflation device 300 of FIG. 3. Balloon inflation device 610a may require manual user input to flow fluid and dilate the balloon 640a. In some embodiments, balloon inflation device 610a may automatically dilate balloon 640a in response to instructions from computing device 650a. For example, the computing device 650a may automatically determine a rate of fluid flow for balloon inflation device 610a based on user input of balloon parameters (e.g., brand, diameter) and/or a target pressure and/or balloon diameter to maintain during the dilation procedure.

Balloon inflation device 610a, digital dilation monitor 620a, balloon catheter 630a, and balloon 640a may all be fluidly coupled to allow fluid flow from the balloon inflation device 610a to the balloon 640a. Fluid may be any suitable fluid for endoscopic dilation, including but not limited to saline. Some or all components may be connected with tubing of any suitable diameter to allow fluid flow. Tubing between different components may be of different diameters. Tubing may be ⅛^{th} of an inch in diameter, in some embodiments. Connections between components and the tubing may be made in any suitable way, including but not limited to national pipe thread (NPT) connections.

The balloon catheter 630a may be any suitable balloon catheter device, including but not limited to a balloon catheter such as balloon catheter 200 depicted in FIG. 2. The balloon 640a, which may be fluidly coupled to balloon catheter 630a, may be of any suitable dimension chosen by the user (e.g., a physician) for the endoscopic dilation procedure. As a non-limiting example, the balloon 640a may be 12, 13.5, and 15 millimeters in diameter.

The digital dilation monitor 620a may be any suitable digital device for monitoring one or more properties of the balloon 640a. As discussed herein, the properties monitored by digital dilation monitor 620a may include properties such as the volume and pressure of fluid flowing to the balloon. In some embodiments, the digital dilation monitor 620a may be a digital dilation monitor such as digital dilation monitor 400 depicted in FIG. 4. As shown in FIG. 6A, the digital dilation monitor 620a may be fluidly coupled in line between the balloon inflation device 610a and balloon catheter 630a in order to monitor the volume and pressure of fluid in the balloon 640a.

The endoscopic dilation monitor 620a may also be electronically coupled to computing device 640a in any suitable manner (e.g., via a wired or wireless connection). The endoscopic dilation monitor 620a may be electronically coupled to computing device 140a through a universal serial bus (USB) connection, in some embodiments. The digital dilation monitor 620a may send information about the measured properties of the balloon 640a, such as fluid flow, pressure, and/or volume, to computing device 650a. In some embodiments, computing device 650a may send instructions to digital dilation monitor 620a (e.g., specifying properties to be measured, units of measurement, duration of measurement, or any other suitable settings for the digital dilation monitor).

FIG. 6B depicts, schematically, an illustrative endoscopic dilation system 600b, according to some embodiments. In FIG. 6B, a depiction of the physical configuration of an exemplary endoscopic dilation system is provided. As shown in the figure, the endoscopic dilation system 600b may include a balloon inflation device 610b, a balloon catheter 630b fluidly coupled to balloon inflation device 610b, and a balloon 640b fluidly coupled to balloon catheter 630b, as described herein at least with respect to FIG. 6A. Although not depicted in this illustration, the balloon catheter 630b and the balloon 640b may be configured to pass through the channel of an endoscope (e.g., such as the endoscope of FIG. 1) into the esophagus of a patient.

In endoscopic dilation system 600b, a digital dilation monitor 620b may be fluidly coupled in line between balloon inflation device 610b and balloon catheter 630b. The digital dilation monitor 620b may be electronically coupled to computing device 650b in any suitable manner, including but not limited to a USB connection.

Computing device 650b may be any suitable computing device including but not limited to a laptop, a tablet, or other portable computing device, as described herein at least with respect to FIG. 11. Computing device 650b, as well as the other components of the endoscope dilation system 600b, may be mounted on a stand 660b. Stand 660b may be, for example, an Ergotron SV10. Mounting the components of endoscopic dilation system 600b on stand 660b may make the endoscope dilation system 600b more portable and easier to use.

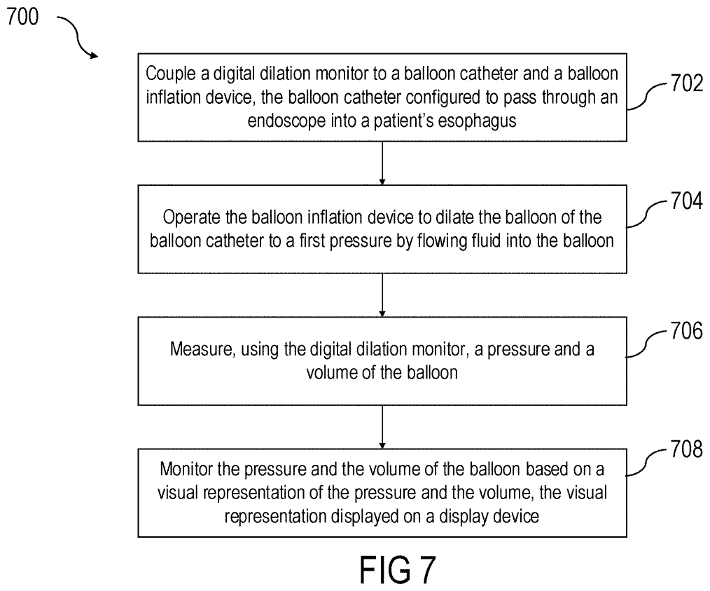

FIG. 7 is a flow diagram illustrating a method 700 for operating an endoscopic dilation system, such as endoscopic dilation system 600a of FIG. 6A, in accordance with some embodiments of the technology described herein. Method 700 may be carried out by a human operator (e.g., a physician, nurse, or technician), or may be performed at least in part by a processor of a computing device (see, e.g., FIG. 11).

In act 702 of method 700, the balloon inflation device (e.g., 610a), digital dilation monitor (e.g., 620a), balloon catheter (e.g., 630a), and balloon (e.g., 640a) may be fluidly coupled so that fluid can flow from the balloon inflation device to the balloon. This may comprise, for example, attaching tubing connections between the various components to be fluidly coupled, as described herein at least with respect to FIGS. 1-6. In act 702, the digital dilation monitor may also be electronically coupled to a computing device (e.g., 650a), as described elsewhere herein. As noted in the figure, the balloon catheter of act 702 may be configured to pass through an endoscope into a patient's esophagus. However, the acts of method 700 are not required to happen while the endoscope is within the esophagus of the patient. For example, the acts of method 700 may be carried out in order to test the functionality of the endoscopic dilation system outside the human body.

In act 704 of method 700, the balloon inflation device may be operated to dilate the balloon to a first pressure. The first pressure may be, for example, a target pressure. A user of the system may provide such a target pressure (and/or other information about the balloon, such as balloon brand, diameter, etc.) as input into the computing device (e.g., via user interface, such as user interface 900 of FIG. 9). Dilation of the balloon with the balloon inflation device may occur manually (e.g., as user pulls a trigger, pushes a button, or operates a crank to activate fluid flow) or automatically (e.g., as the computing device controls the balloon inflation device to flow fluid to the balloon). The computing device may determine a rate of fluid flow based on the user input about the balloon, in order to facilitate automatic inflation of the balloon. The computing device may also determine an upper control limit pressure and lower control limit pressure based on a target pressure provided by the user. The upper control limit pressure and lower control limit pressure may define a range of pressures around the target pressure. For example, this range of pressures may be within 1%, 5%, 10%, 15%, 20%, 25%, or 30% of the target pressure.

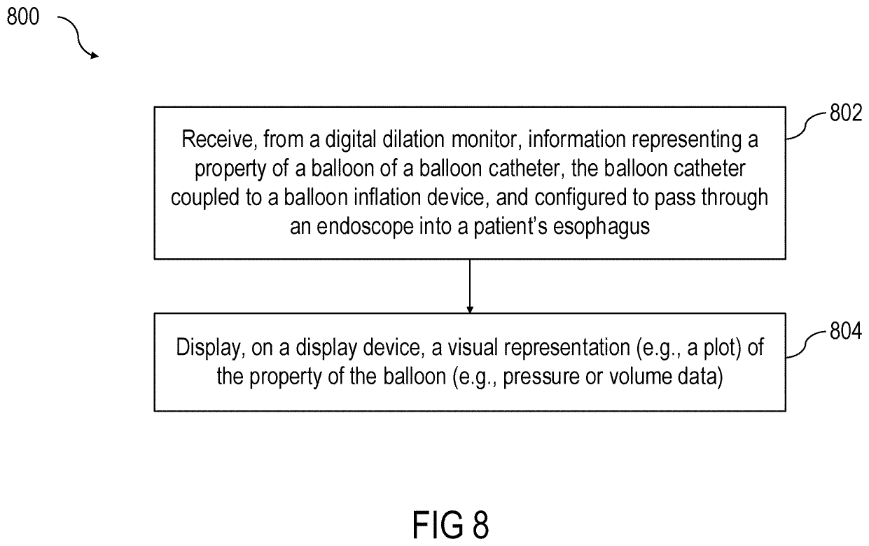

In act 706 of method 700, the pressure and/or volume of the balloon may be measured using the digital dilation monitor. As noted herein at least with respect to FIG. 8, in some cases, properties of the balloon other than pressure and/or volume may be measured using the digital dilation monitor. As such, it should be appreciated that method 700 may be performed using any such properties measured using the digital dilation monitor, mutatis mutandis. In some embodiments, the digital dilation monitor may be configured to perform measurements automatically, without human intervention. In some embodiments, the digital dilation monitor may require manual setup (including, for example, activating the digital dilation monitor by operating a power switch or button, selecting appropriate properties to be measured via an interface of the digital dilation monitor, etc.). In some embodiments, measuring the pressure and/or volume of the balloon may occur while the balloon is being dilated in real time. In some embodiments, the pressure and/or volume of the balloon may be measured before dilation occurs, or after dilation is complete.

In act 708 of method 700, the pressure and/or the volume of the balloon may be monitored based on a visual representation of the pressure and/or volume displayed on a display device. For example, the digital dilation monitor may send information representing the pressure and/or volume of the balloon to the computing device (e.g., via a wired or wireless connection, as described elsewhere herein), which the computing device may process in order to display a visual representation of the information on a display device (e.g., a laptop screen, or any other appropriate display device). The pressure and/or volume data from the digital dilation monitor may be displayed, for example, as part of a user interface (see, e.g., FIG. 9) in order to allow a user to monitor the pressure and/or volume of the balloon. Monitoring the pressure and/or volume of the balloon may include monitoring a plot of the pressure and/or volume information (e.g., as pressure and/or volume curve(s) over time). Other visual representations of the pressure and/or volume information may additionally or alternatively include numeric representations, animated depictions (e.g., of the balloon expanding, of the plotted information as it is being collected in real time, etc.), and/or other types of graphs or charts.

Monitoring of the pressure and/or volume of balloon in act 708 may further comprise monitoring the functionality of the balloon inflation device. For example, over time, the balloon inflation device may degrade in functionality (e.g., not be able to stably maintain pressure and/or volume in the balloon catheter, allow for a negative flow of fluid out of the balloon, etc.). Monitoring the functionality of the balloon inflation device may allow a user to know when to replace the balloon inflation device, for example.

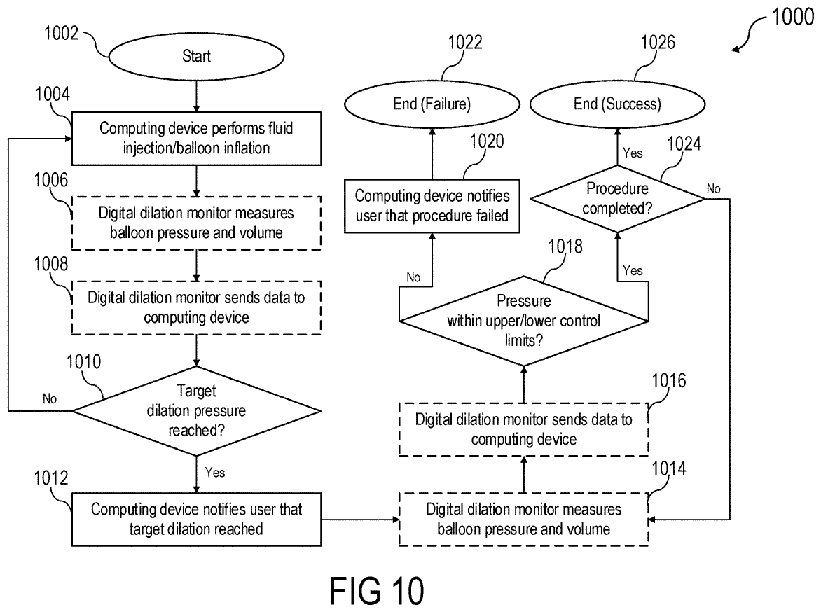

Monitoring of the pressure and/or volume of the balloon may further comprise monitoring whether the pressure of the balloon remains between the upper control limit pressure and lower control limit pressure. In some embodiments, the computing device may additionally or alternatively monitor the pressure and/or volume of the balloon, as described herein at least with respect to FIG. 10. If the computing device determines that the pressure of the balloon exceeds the upper control limit pressure and/or falls below the lower control limit pressure, computing device may alert the user of an anomaly. Alerting the user may, for example, include displaying a message, displaying a light, and/or producing an audible alert.

Monitoring of the pressure and/or volume of balloon may further comprise selecting to save information representing the pressure and/or volume of the balloon to a non-transitory computer readable storage medium associated with the computing device (see, e.g., FIG. 11). In some embodiments, the user may select to save the information via the user interface displayed on the display device. In some embodiments, the information may be saved automatically (e.g., upon completion of the dilation procedure). The stored information may be further manipulated by the computing device in order to calculate further statistics, such as power and energy of the system. In particular, the power and energy of the system may be calculated at each point in time for which pressure and/or volume data were collected. This may provide, in addition to pressure and/or volume curves, power and/or energy curves depicting the power and/or energy over time.

In some embodiments, the stored information may be compared across two or more endoscopic dilation procedures. In some embodiments, stored plots (e.g., pressure, volume, power, or energy curves) may be compared across endoscopic dilation procedures. For example, pressure curves may be compared between endoscopic dilations procedures utilizing different types of balloon inflation devices (e.g., crank operated vs trigger operated balloon inflation devices) so as to analyze (e.g., with a computing device or manually) their efficacy. Such techniques may include, for example, analyzing a static and/or dynamic pressure associated with the pressure curves for the various balloon inflation devices.

In some embodiments of method 700, dilating the balloon at act 704 may further comprise conditionally dilating the balloon if the desired pressure is not maintained after reaching the target pressure, as determine in acts 706 and 708. Conditionally dilating the balloon may comprise continuing to dilate the balloon if the pressure and/or volume curves of the balloon are the same inside the body of the patient as they would be if measured outside of the body of the patient. Such a pressure and/or volume curve may indicate that the stricture of the esophagus is not placing pressure on the balloon. Conditionally dilating the balloon may additionally or alternatively comprise not dilating the balloon further if there is a drop in static pressure after the target pressure is reached in act 704. Such a drop in static pressure may indicate mucosal damage to the patient. Further, conditionally dilating the balloon may comprise ending the dilation process if wide swings in pressure are observed in response to small changes in volume of the balloon. Such swings in pressure may indicate that the stricture may not respond to dilation and may require different treatment methods.

FIG. 8 is a flow diagram illustrating a computer-implemented method 800 for displaying information from an endoscopic dilation system, in accordance with some embodiments of the technology described herein. The acts of method 800 may be carried out by computing device acting within an endoscopic dilation system, as described herein at least with respect to FIGS. 6A-6B. The acts of method 800 may correspond to the acts carried out by the computing device during act 708 of method 700.

At act 802 of method 800, the computing devices may receive information representing a property of a balloon (e.g., the pressure and/or volume of the balloon, as in method 700) from the digital dilation monitor. As noted in the figure, the balloon may be part of a balloon catheter, coupled to a balloon inflation device and configured to pass through an endoscope into a patient's esophagus. The information received in act 802 may be received via a communication channel (e.g., a wired or wireless connection, such as a USB connection, a WiFi or Bluetooth connection, or any other suitable connection). The information received in act 802 may be stored on at least one non-transitory storage medium associated with the computing device, as described herein with respect to act 708 of method 700.

At act 804 of method 800, the computing device displays, on a display device, a visual representation of the property of the balloon. As noted in the figure, this may comprise plotting pressure and/or volume data (or information representing any other property of the balloon) as described herein with respect to method 700. In order to produce the visual representation of the property of the balloon, the computing device may perform further operations upon the information received from the digital dilation device. This may comprise, for example, converting the received information to appropriate units, organizing the received information into particular data structures and/or formats for further processing, and processing the received information for display (e.g., by determining a scale of the axes of a plot, performing smoothing, rounding, approximation, or filtering operations, or any other suitable processing techniques).

FIG. 9 depicts an illustrative user interface 900 for an endoscopic dilation system, in accordance with some embodiments of the technology described herein. The user interface 900 may be displayed on a display device by a computing device, for example as part of the techniques described herein at least with respect to FIGS. 7 and 8. A user may interact with the user interface 900 in any suitable manner, including, for example, with a mouse, a keyboard, a touch screen, or any other suitable means of input. The user interface 900 may display visual representation(s) of one or more properties of a balloon of a balloon catheter based on information received from a digital dilation monitor, as described herein at least with respect to FIGS. 7 and 8.

As shown in the figure, the user interface 900 includes a volume plot 902. In this example, the volume plot 902 indicates a total volume of the balloon in cubic centimeters along the x-axis, with time being plotted along the y-axis. Volume data may be plotted in plot 902 in real time (e.g., as the balloon of the balloon catheter is being inflated). The axes of plot 902 may update automatically as volume data is plotted over time, or they may be updated manually by the user.

The user interface 900 also includes a pressure plot 904. In this example, the pressure plot 904 indicates a pressure of the balloon in in kPaG along the x-axis, with time being plotted along the y-axis. As shown in the figure, the pressure plot may include text indicating a pressure which should not be exceeded during balloon dilation (e.g., “Do not exceed 1380 kPa (200 PSI)”).

As shown in the figure, user interface 900 includes a variety of other elements with which a user may interact. For example, the user interface 900 may include a number of buttons, input fields, and checkboxes operable by the user. Non-limiting examples of elements that may appear as part of user interface 900 include: a reset totalizer button (e.g., for total volume calculations); a tare flow button (e.g., to zero a flow measurement); graphing options, including an adjust scale button, and checkboxes to specify whether graphing is enabled and whether the pressure graph is being automatically scaled; data logging options, including checkboxes to specify what information is to be saved, an input field to enter a directory to which the data is to be saved, and a log data button (e.g., to save the data to the non-transitory computer-readable storage medium); input fields to enter patient data, communication (COM) port settings, balloon inflated O.D. (in millimeters), and timing options (e.g., desired time between readings in milliseconds, etc.); buttons to save or load a setup of the user interface; and buttons to save or erase defaults (e.g., default settings) of the user interface.

FIG. 10 is a flow diagram illustrating a computer-implemented method 1000 for automatically operating an endoscopic dilation system, in accordance with some embodiments of the technology described herein. The acts of method 1000 may be carried out by a computing device acting as part of the endoscopic dilation system, as described herein at least with respect to FIGS. 6-8.

Method 1000 begins at act 1002. Method 1000 may be initiated by user input (e.g., via a user interface such as user interface 900) or may begin automatically.

At act 1004, the computing device performing method 1000 may operate a balloon inflation device to inject fluid into a balloon of a balloon catheter, thereby inflating the balloon. As noted elsewhere herein, the computing device may calculate a flow speed for the fluid as part of inflating the balloon. This calculation may be performed based on balloon size (e.g., as provided by the user), and/or a size of the stricture of the esophagus.

At act 1006, a digital dilation monitor of the endoscopic dilation system may measure balloon pressure and/or volume (or any other suitable property of the balloon, as described herein). At act 1008, this data may be sent to the computing device (e.g., via a wired or wireless communication channel, as described herein). The computing device may be monitoring the information received from the digital dilation monitor in order to detect a pressure drain corresponding to an expansion of the esophagus.

At act 1010, the computing device checks, based on the information received from the digital dilation monitor, whether a target dilation pressure of the balloon has been reached. The target dilation pressure may be a value set by a user (e.g., a physician) via a user interface, or a value set automatically by the computing device (e.g., based on the size or the balloon and/or a size of the stricture of the esophagus). The computing device may be configured to detect a sharp decline in pressure, indicated that the pressure drain has been reached.

If the target dilation pressure has not been reached at act 1010, then method 1000 returns to act 1004. The computing device may continue to inflate the balloon as shown in acts 1004 to 1010, until the target pressure has been reached. Once the target pressure has been reached, the computing device may automatically set an upper and lower control limit for the pressure. The upper and lower control limits may be based on a pressure the physician is trying to maintain during dilation (referred to herein as a nominal pressure). The nominal pressure may be dependent upon the pressure drop and the elastic response of the esophagus.

If the target pressure has been reached at act 1010, then method 1000 proceeds to act 1012. At act 1012, the computing device notifies the user that the target dilation pressure has been reached. This may comprise displaying text on a display device, activating an indicator light, playing an audible signal, or any other suitable means of notifying the user.

At acts 1014 and 1016, the digital dilation monitor may continue to measure and send data regarding the balloon (e.g., pressure and volume data) to the computing device, as in acts 1006 and 1008 of method 1000. At act 1018, the computing device checks, based on the information received from the digital dilation monitor, whether the pressure of the balloon is still within the upper and lower control limits.

If the pressure fails to remain within the control limits (e.g., by exceeding the upper control limit or falling below the lower control limit), then method 1000 proceeds to act 1020, where the user is notified that the dilation procedure has failed. The user may notified in any suitable way, as described herein at least with respect to act 1012. At act 1022, the method 1000 ends with failure.

If the pressure remains within the control limits at act 1018, then method 1000 proceeds to act 1024, where the computing device checks whether the dilation procedure has been completed. The computing device may automatically detect that the dilation procedure is complete (e.g., based on the data received from the digital dilation monitor), or the user may specify, in advance or during the dilation procedure, when the procedure is complete. If the procedure is not complete at act 1024, then method 1000 returns to act 1014, allowing the properties of the balloon to be continuously measured and monitored by the computing device in acts 1014 to 1018. If the procedure is complete at act 1024, then method 1000 proceeds to act 1026, where it ends with success. In some embodiments, the computing device may automatically save information from the method 1000, whether it ends in success or failure, to a non-transitory computer-readable storage medium. For example, pressure and/or volume information may automatically be logged to a comma separated values (CSV) file or a spreadsheet.

FIG. 11 shows, schematically, an illustrative computer 1100 on which any aspect of the present disclosure may be implemented. The computer 1100 includes a processing unit 1101 having one or more processors and a non-transitory computer-readable storage medium 1102 that may include, for example, volatile and/or non-volatile memory. The memory 1102 may store one or more instructions to program the processing unit 1101 to perform any of the functions described herein. The computer 1100 may also include other types of non-transitory computer-readable medium, such as storage 1105 (e.g., one or more disk drives) in addition to 20 the system memory 1102. The storage 1105 may also store one or more application programs and/or resources used by application programs (e.g., software libraries), which may be loaded into the memory 1102.

The computer 1100 may have one or more input devices and/or output devices, such as devices 1106 and 1107 illustrated in FIG. 11. These devices may be used, for instance, to present a user interface. Examples of output devices that may be used to provide a user interface include printers and display screens for visual presentation of output, and speakers and other sound generating devices for audible presentation of output. Examples of input devices that may be used for a user interface include keyboards and pointing devices (e.g., mice, touch pads, and digitizing tablets). As another example, the input devices 1107 may include a microphone for capturing audio signals, and the output devices 1106 may include a display screen for visually rendering, and/or a speaker for audibly rendering, recognized text.

In the example shown in FIG. 11, the computer 1100 also includes one or more network interfaces (e.g., the network interface 1110) to enable communication via various networks (e.g., the network 1120). Examples of networks include a local area network (e.g., an enterprise network) and a wide area network (e.g., the Internet). Such networks may be based on any suitable technology and operate according to any suitable protocol, and may include wireless networks and/or wired networks (e.g., fiber optic networks).

Having thus described several aspects of at least one embodiment of this invention, it is to be appreciated that various alterations, modifications, and improvements will readily occur to those skilled in the art.

Such alterations, modifications, and improvements are intended to be part of this disclosure, and are intended to be within the spirit and scope of the invention. Further, though advantages of the present invention are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semi-custom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Also, the various methods or processes outlined herein may be coded as software that is executable on one or more processors running any one of a variety of operating systems or platforms. Such software may be written using any of a number of suitable programming languages and/or programming tools, including scripting languages and/or scripting tools. In some instances, such software may be compiled as executable machine language code or intermediate code that is executed on a framework or virtual machine. Additionally, or alternatively, such software may be interpreted.

The techniques disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more processors, perform methods that implement the various embodiments of the present disclosure discussed above. The computer-readable medium or media may be transportable, such that the program or programs stored thereon may be loaded onto one or more different computers or other processors to implement various aspects of the present disclosure as discussed above.

The terms “program” or “software” are used herein to refer to any type of computer code or set of computer-executable instructions that may be employed to program one or more processors to implement various aspects of the present disclosure as discussed above. Moreover, it should be appreciated that according to one aspect of this embodiment, one or more computer programs that, when executed, perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Program modules may include routines, programs, objects, components, data structures, etc. that perform particular tasks or implement particular abstract data types. Functionalities of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields to locations in a computer-readable medium that convey relationship between the fields. However, any suitable mechanism may be used to establish a relationship between information in fields of a data structure, including through the use of pointers, tags, or other mechanisms that establish relationship between data elements.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which examples have been provided in FIGS. 7, 8, and 10. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. An endoscopic balloon dilation system comprising:
an endoscope configured to be inserted into an esophagus of a patient;
a balloon catheter comprising a balloon, the balloon catheter configured to pass through the endoscope into the esophagus of the patient;
a balloon inflation device fluidly coupled to the balloon catheter, the balloon inflation device configured to dilate the balloon by flowing fluid into the balloon;
a digital dilation monitor fluidly coupled to the balloon catheter and the balloon inflation device, the digital dilation monitor configured to measure at least one property of the balloon;
at least one processor; and
at least one non-transitory computer-readable storage medium storing processor executable instructions that, when executed by the at least one processor, cause the at least one processor to perform a method comprising:
receiving, from the digital dilation monitor, information representing the at least one property of the balloon; and
displaying, on a display device, a visual representation of the at least one property of the balloon.

2. The endoscopic balloon dilation system of claim 1, wherein the at least one property of the balloon comprises a pressure of the balloon and/or a volume of the balloon.

3. (canceled)

4. The endoscopic balloon dilation system of claim 1, wherein the method further comprises:
storing the information representing the at least one property of the balloon on the at least one non-transitory computer-readable storage medium.

5. The endoscopic balloon dilation system of claim 1, wherein the visual representation of the at least one property of the balloon comprises:
a plot of the pressure of the balloon and/or a plot of the volume of the balloon.

6. (canceled)

7. The endoscopic balloon dilation system of claim 1, wherein the visual representation of the at least one property of the balloon is displayed to a user as part of a user interface.

8. The endoscopic balloon dilation system of claim 1, wherein the user interface allows the user to select to store the information representing the at least one property of the balloon on the at least one non-transitory computer-readable storage medium.

9. The endoscopic balloon dilation system of claim 1, wherein the method further comprises:
calculating an energy of the system at a time based on a pressure and a volume of the balloon at the time.

10. The endoscopic balloon dilation system of claim 9, wherein the method further comprises:
calculating a power input of the system at the time based on the energy and the time.

11. The endoscopic balloon dilation system of claim 1, wherein the method further comprises:
receiving, from a user, a target pressure for the balloon.

12. The endoscopic balloon dilation system of claim 1, wherein the method further comprises:
establishing, based on the target pressure, an upper and/or lower control limit pressure for the balloon.

13. (canceled)

14. The endoscopic balloon dilation system of claim 12, wherein the method further comprises:
notifying the user if the pressure exceeds the upper control limit pressure for the balloon.

15. The endoscopic balloon dilation system of claim 12, wherein the method further comprises:
notifying the user if the pressure is below the lower control limit pressure for the balloon.

16. The endoscopic balloon dilation system of claim 15, wherein notifying the user comprises displaying a visual indicator to the user via the user interface.

17. The endoscopic balloon dilation system of claim 1, wherein the method further comprises:
conditionally operating the balloon inflation device to dilate the balloon by flowing fluid into the balloon.

18. The endoscopic balloon dilation system of claim 1, wherein the balloon inflation device is conditionally operated based on the information representing the at least one property of the balloon.

19. The endoscopic balloon dilation system of claim 1, wherein the balloon inflation device is conditionally operated based on a target pressure.

20. The endoscopic balloon dilation system of claim 1, wherein the digital dilation monitor is configured to measure the at least one property of the balloon in real time as the balloon is being dilated.

21. The endoscopic balloon dilation system of claim 1, wherein the visual representation of the at least one property the balloon is displayed in real time as the balloon is being dilated.

22. A method of performing endoscopic balloon dilation using a digital dilation monitor, the method comprising:
fluidly coupling the digital dilation monitor to a balloon catheter and a balloon inflation device, the balloon catheter configured to pass through an endoscope into an esophagus of a patient;
operating the balloon inflation device to dilate the balloon of the balloon catheter to a first pressure by flowing fluid into the balloon;
measuring, using the digital dilation monitor, a pressure and a volume of the balloon; and
monitoring the pressure and the volume of the balloon based on a visual representation of the pressure and the volume, the visual representation displayed on a display device.

23.-28. (canceled)

29. At least one non-transitory computer-readable storage medium storing processor executable instructions that, when executed by at least one processor, cause the processor to perform a method comprising:
receiving, from a digital dilation monitor, information representing at least one property of a balloon of a balloon catheter, the digital dilation monitor fluidly coupled to the balloon catheter and a balloon inflation device, the balloon catheter configured to pass through an endoscope into an esophagus of a patient, the balloon inflation device configured to dilate the balloon by flowing fluid into the balloon; and
displaying, on a display device, a visual representation of the at least one property of the balloon.

30.-49. (canceled)
